(19) United States
(12) Patent Application Publication
(10) Pub. No.: US 2002/0177899 A1
(43) Pub. Date: Nov. 28, 2002

(54) Method of loading a stent on a delivery catheter

(76) Inventors: Jay J. Eum, Irvine, CA (US); Paul W. Mikus, Irvine, CA (US); Gregory L. Kelly, Irvine, CA (US)

Correspondence Address: Lawrence N. Ginsberg, Endocare, Inc., 201 Technology Drive, Irvine, CA, 92618, US

(21) Appl. No.: 10/190,859

(22) Filed: Jul. 8, 2002

Related U.S. Application Data
(62) Division of application No. 09/433,695, filed on Nov. 3, 1999, now abandoned.

(51) Int. Cl. A61F 2/04

(52) U.S. Cl. 623/23.7; 623/1.19

(57) ABSTRACT

A method of loading a shape memory, superelastic (or pseudoelastic) stent onto an insertion catheter by cooling the stent to its martensite state with a spray of refrigerant, cold gas, or expanding gas. The stent may then be loaded onto the delivery catheter without the force necessary to deform the stent through the formation of stress induced martensite.

### FIELD OF THE INVENTIONS

This invention relates to stents, and more generally to a method for preparing nitinol medical devices for insertion into the body.

### BACKGROUND OF THE INVENTIONS

Various implantable medical devices such as stents, bone clips, vena cava filters, etc. are most easily and safely inserted into the body if they are first compressed into a small configuration, then inserted into the body and expanded. Stents, for example, are compressed to fit into a catheter which is then inserted into the body vessel such as a coronary artery or the urethra, then expanded and released. An example is shown in our patent, Mikus, Urological Stent Therapy and Method, U.S. Pat. No. 5,830,179, the disclosure of which is hereby incorporated by reference, which shows a helical stent made of nitinol, compressed and inserted into a catheter for placement into the prostatic urethra. Various other patents show stents of differing configurations and temperature regimens. Jervis, Medical Devices Incorporating SIM Alloy Elements, 4,665,906 (May 19, 1987) discloses a nitinol stent which is pseudoelastic at body temperature and unwinds into the deployed configuration through superelasticity. Jervis specifically calls for loading the stent into a delivery catheter by deforming the stent through the formation of “stress induced martensite.” In order for nitinol to support the formation of stress induced martensite, it must be at a temperature within the range in which martensite may be formed through the application of stress (deforming force). While deforming the stent through the formation of stress induced martensite may have benefits, it requires stress, or force, and that force is substantial compared to the strength of the other components in the system. Also, the deformed SIM device in the SIM temperature range always reverts to its memorized shape, so that it will not stay in any one configuration during handling if it is handled in the SIM temperature range. By cooling the stent to a temperature at which stress induced martensite and pseudoelastic behavior cannot occur, assembly of the stent and delivery system is facilitated because it requires less force to deform the stent and the stent remains in a stable deformed shape.

### SUMMARY

In order to reduce the force necessary to load nitinol stents onto an insertion catheter, the stent is cooled to temperatures well below the martensite state of the alloy making up the stent. Because the stent is completely martensitic and no austenite remains in the stent, it is pliable and ductile, and easily deformed as necessary for loading into an insertion catheter. Since the stent need not be deformed through the formation of stressed induced martensite, much less force is required to deform the stent. Cooling is accomplished in various embodiments of the method by spraying the stent with a freeze spray, or an expanding gas, so that the stent is not wetted during handling.

### BRIEF DESCRIPTION OF THE DRAWINGS

FIG. 1 illustrates the method of cooling the stent prior to insertion into an insertion catheter.

FIG. 2 is a graphical illustration of the stent's behavior in response to temperature changes.

### DETAILED DESCRIPTION OF THE INVENTIONS

The stent is prepared for loading merely by cooling. The stent should be washed and dried prior to cooling, deformation and insertion into the delivery catheter. An ultrasonic bath in a dilute detergent and water solution is suitable. Prior to depositing the stent in the bath, the ultrasonic power source is energized for several minutes to drive any absorbed gas out of the solution. The stent is then bathed in the ultrasonic bath, with the ultrasonic power source energized, for several minutes, and then rinsed to remove the detergent.

The stent is cooled to a temperature below the T_{mf} temperature prior to deformation and insertion into the delivery catheter. This is the temperature at which any and all austenitic metal in the stent has been converted to martensite. The cooling may be accomplished by performing the entire stent loading procedure in a refrigerated clean room or bathing the stent in a cold water or fluid bath maintained at a temperature below the T_{mf} of the stent metal. More economically, the stent is cooled with a gaseous or liquid spray. The spray may be a rapidly evaporating liquid which cools as it evaporates, such as HFC-134a. These compounds are typically used for cooling electronics, as a troubleshooting aid, or for protection from heat. Other refrigerants such as freon may be used. The spray may also be comprised of dry compressed air, nitrogen gas, carbon dioxide or other gas that cools when expanding from a nozzle. Cold water may be used if additional steps are taken to prevent the water from entering and/or remaining in the delivery system and creating a risk of contamination. Other liquids which evaporate quickly or which do not encourage biological contamination may be used (alcohol, for example). Where refrigerants, oxygen displacing gas, or toxic cooling fluids are used for the spray, an appropriate containment area such as a glove box should be used. The cooling fluid may be maintained within the glove box or purged safely from the glove box.

A suitable cooling medium is available in the form of a spray sold under the name Envi-Ro-Tech Freezer by Tech Spray of Amarillo, Tex. This formulation has proven to be non-cytotoxic when sprayed onto stents. It evaporates quickly and leaves no trace chemicals on the stent. The chemical compound is 1,1,1,2-tetraflouroethene, and it is safe for use in a well-ventilated area or in a glove box.

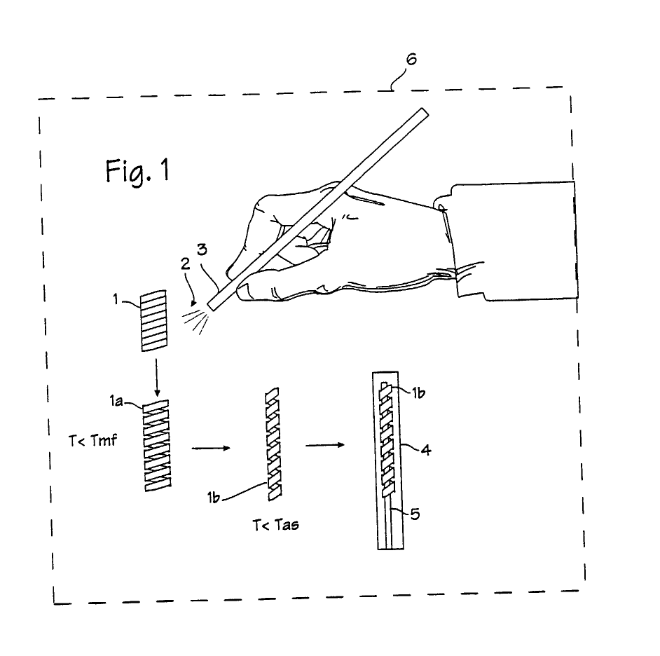

FIG. 1 illustrates the method of cooling and deforming a stent for loading into an insertion catheter. The stent 1 is comprised of a shape memory metal such as nitinol, and has a characteristic martensite temperature zone, austenite temperature zone, and a transition temperature zone in between in which the shape memory metal is comprised partially of both martensite and austenite. The stent 1 is sprayed with a cooling fluid 2. The fluid is dispensed from spray nozzle 3, which may be hand held and manipulated to spray substantially the entire surface of the stent. Preferably, the assembler wears gloves when handling the coolant and the stent, both to avoid freezing the skin and to avoid warming the stent during manipulation. The stent cools upon being sprayed, either through evaporative cooling of the cooling fluid, or because the cooling fluid is cold. Spraying and cooling are continued until the stent is fully cooled to martensite. The stent 1 is transformed to martensite upon cooling, and becomes pliable and soft. In the case of the helical stent illustrated, the coils will become loose and floppy, depicted as the stent in condition 1a. Thereafter, the stent may be deformed to a small diameter condition, depicted as the stent in condition 1b, and loaded into an insertion catheter 4, mounted on an inner sheath or rod 5. During the handling process, it is preferable to maintain the stent at a temperature below the Tₐₛ of the nitinol alloy making up the stent. The ambient atmosphere in the workplace 6 may be maintained below Tₐₛ, which is quite easy for any alloy with a Tₐₛ above room temperature 68°-72° F. Where Tₐₛ is below room temperature, the workplace may be air-conditioned to a temperature below Tₐₛ or at a temperature below room temperature (but above Tₐₛ) in order to slow warming of the stent to Tₐₛ. If ambient temperature in the workplace is above Tₐₛ, stent deformation may be done rapidly before the stent warms to ambient temperatures. In cases of very low Tₐₛ, the stent may be cooled and manipulated in a refrigerated glove box. Those familiar with stents will appreciate that there are many designs for insertion catheters and delivery systems which can be used, and many forms of stents, such as coiled stents, braided stents, slotted expanding stents, etc. which, when comprised of a shape memory material, can be cooled and loaded in this manner. The process can be used for any medical device, such as vena cava filters, bone staples, etc. which require deformation prior to insertion into the body.

Nitinol is a readily available material for the stent. Accordingly, the stent preferably is comprised of nitinol, and it is fabricated with an Austenite Finish Temperature (T_{af}) of 25-45° C. (preferably in the range of 30° C.±5° (86°±9° F.)) and an Austenite Start Temperature (Tₐₛ) of 0 to 20° C. (preferably in the range of 10° C. (50° F.)) or higher. The freeze spray method readily cools the stent to −10° C. (10° F.), eliminating the potential for creating stress induced martensite, and providing a lengthy period for manipulation even where ambient temperature is room temperature. Thus, during handling and loading, the stent will consist entirely of nitinol in its thermally induced martensite form.

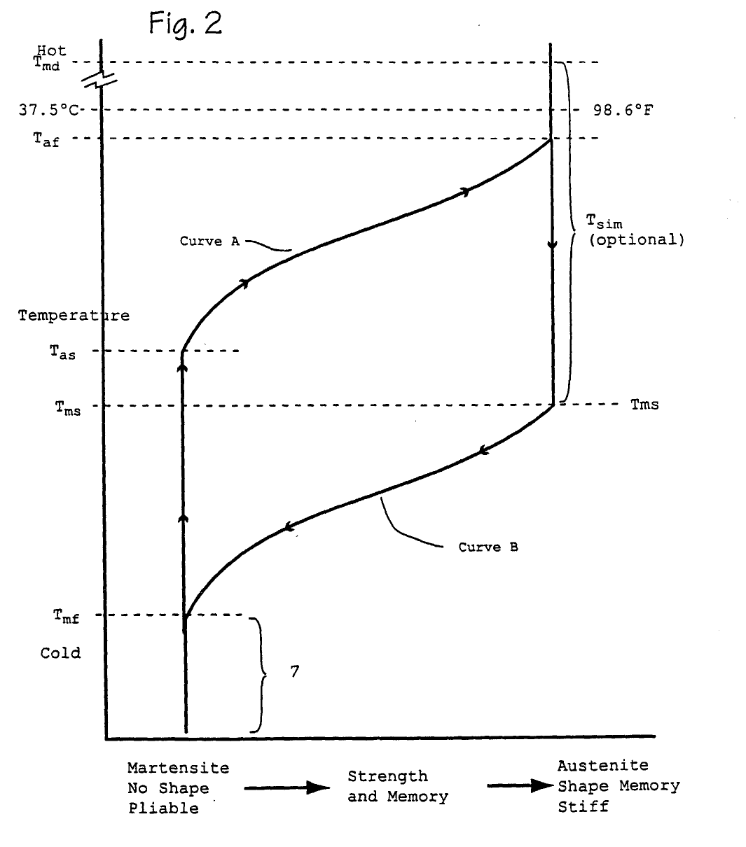

FIG. 2 illustrates the metallurgical behavior of the stent. The stent is made of a shape memory alloy with a martensite state at cold temperature and an austenite state at high temperature, as is characteristic. Nitinol, comprised mostly of nickel and titanium is the most common shape memory alloy, however numerous alloys behave in similar fashion. At low temperature, the stent is in its martensite state, and is very pliable and has no memorized shape and has very little strength. This is shown on the graph on curve A. As temperature rises, the metal starts to convert to austenite at a certain temperature (determined by a variety of factors, including composition of the alloy, readily controlled in the art of shape memory alloys) called the austenite start temperature, Tₐₛ. The metal becomes stronger, stiffer, and reverts to its memorized shape as temperature increases to T_{af}. At the austenite finish temperature, T_{af}, the alloy has completely reverted to austenite, has recovered its memorized shape (unless restrained), and is stiff like spring steel. Above T_{af}, temperature increases do not affect the shape or shape memory behavior of the metal, except that above T_{md}. no stress induced martensite can be formed due to the high temperature of the alloy. Upon cooling, the metal reverts to the martensite state, but this does not occur exactly in reverse. The temperature at which reversion to martensite occurs upon cooling is lower than the temperature at which martensite-to-austenite conversion occurs on heating. As shown in the graph, upon cooling to the martensite start temperature, Tₘₛ, which in this case is below body temperature, the metal starts to become pliable. Further cooling to the martensite finish temperature T_{mf} results in the complete conversion of the alloy to the soft, pliable martensite state. Superelastic behavior occurs around the region of Curve B below T_{md}, and above Tms if the alloy was first at a high temperature austenite state. The metal may be substantially bent (deformed) but still spring back to its memorized shape. The deformation is accommodated in the metal through the formation of stress induced martensite, which in this temperature range reverts back into the austenite state upon removal of the stress only if the stent is initially austenitic. This region is shown on the graph as Tₛᵢₘ, which varies from alloy to alloy and might not be present in some alloys. This region does not extend to portion 7 of the curve, where there is no austenite in the metal, the metal is entirely martensitic, and no martensite may be stress induced. If the alloy is initially in the martensite state, superelastic behavior will not occur until the alloy is heated to a temperature above Tₐₛ (on curve A), so that the metal may be substantially bent (deformed) in this region and will not spring back to its memorized shape. In the region from T_{mf} and below (region 7) to Tₐₛ, the alloy cannot form stress induced martensite, and austenite will not form. In this temperature range, deformation of the stent will result in a stable shape, since shape change occurs only through the formation of austenite. The stents used in the new method are cooled to the temperature range below T_{mf}, in region 7. They are then deformed, while they remain in the region below Tₐₛ, so that no shape recovery occurs, no austenite is formed, and no stress induced martensite may be formed. They are then placed in an insertion catheter and stored for use. In use, the insertion catheter is inserted into the body to the point where the stent is to be place, and the stent is then released to remain in the body. The stents may be pseudoelastic at body temperature, so that they revert to their memorized shapes upon warming to body temperature, or they may not be pseudoelastic at body temperature and require additional heating to the austenite transition temperature. Alloys and devices incorporating these characteristics may be manufactured according to known methods in the art of metallurgy.

The method described above may be used for stents or any other medical device which requires deformation prior to insertion and implantation into the body. The devices may be pseudoelastic at body temperature, and thus isothermally transform from the deformed state to the memorized shape without additional heat sources, or activated by heating to a shape memory transition temperature. The temperature ranges related above may be manipulated and altered in the fabrication of the nitinol or other shape memory material. The insertion catheter is one of many restraining means that can be used to hold the medical device in the small condition and hold the device for insertion into the body. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A method for loading a stent on an insertion catheter, said method comprising the steps of:
providing an insertion catheter adapted to hold a stent in a small diameter condition;
providing a stent comprised of shape memory material, pseudoelastic material or superelastic material characterized by a conversion to a low temperature state in which the stent is relatively pliable when the stent is at a low temperature range and a high temperature state in which the stent is relatively stiff when the stent is in a high temperature range;
spraying the stent with a fluid, said fluid being at a temperature within the low temperature range, until the stent is cooled to the low temperature range, thereby making the stent pliable;
deforming the stent while the stent remains within the low temperature range as necessary to load the stent onto the insertion catheter in a small diameter condition.

2. The method of claim 1, wherein the fluid used is a gas.

3. The method of claim 1, wherein the fluid used is an expanding gas.

4. The method of claim 1, wherein the fluid used is a refrigerant.

5. The method of claim 1, wherein the fluid used is a freeze spray.

6. The method of claim 1 further comprising:
maintaining the ambient atmosphere around the stent at a temperature below the high temperature range.

7. A method for loading a stent on an insertion catheter, said method comprising the steps of:
providing an insertion catheter adapted to hold a stent in a small diameter condition;
providing a stent comprised of nitinol characterized by a conversion to a martensite state when the stent is at a low temperature range below the Tₘₛ of the nitinol, said conversion to the martensite state being complete when the nitinol is cooled to a temperature range below the T_{mf} of the nitinol, and conversion to an austenite state when the stent is in a temperature range above Tₐₛ of the nitinol;
spraying the stent with a fluid, said fluid adapted to cool the stent to a temperature below T_{mf}, until the stent is cooled to a temperature below T_{mf}, thereby converting the stent to thermally induced martensite;
deforming the stent while the stent below Tₐₛ and the nitinol in the stent is completely comprised of thermally induced martensite;
loading the stent onto the insertion catheter in the deformed condition.

8. The method of claim 6, wherein the fluid used is a gas.

9. The method of claim 6, wherein the fluid used is an expanding gas.

10. The method of claim 6, wherein the fluid used is a refrigerant.

11. The method of claim 6, wherein the fluid used is a freeze spray.

12. The method of claim 6 further comprising:
maintaining the ambient atmosphere around the stent at a temperature below Tₐₛ of the nitinol.

13. A method of deforming a nitinol stent for loading the stent onto an insertion catheter without deforming the stent through the formation of stress induced martensite, said method comprising:
spraying the stent with a cooling fluid until the stent is cooled to a temperature range where it is completely comprised of martensite and incapable of supporting the formation of stress induced martensite;
deforming the stent at a temperature below the temperature at which austenite begins to form in the nitinol in the stent.

14. A method of installing a pseudoelastic shape-memory alloy medical device within a mammalian body, wherein the pseudoelastic shape-memory alloy medical device displays reversible stress-induced martensite at body temperature, the method comprising:
deforming the medical device into a deformed shape different from a final shape, said deforming occurring without the formation of stress-induced martensite;
restraining the deformed shape of the medical device by the application of a restraining means;
positioning the medical device and restraining means within the body;
removing the restraining means;
isothermally transforming the device from the deformed shape into the final shape.
